(19) United States
(12) Patent Application Publication
(10) Pub. No.: US 2026/0306840 A1
(43) Pub. Date: Oct. 8, 2026

(54) Multimodal Sensors From Wearables and Implants to Improve Diagnosis and Efficiency of Physiologic Monitoring

(71) Applicant: BIOTRONIK SE & Co. KG, Berlin (DE)

(72) Inventors: R. Hollis WHITTINGTON, Portland, OR (US); Dirk MUESSIG, West Linn, OR (US); Ravi Kiran Kondama REDDY, Portland, OR (US); Patrick L. PARISH, Portland, OR (US); Daniel YOUNG, Portland, OR (US)

(73) Assignee: BIOTRONIK SE & Co. KG, Berlin (DE)

(21) Appl. No.: 19/481,802

(22) PCT Filed: May 15, 2024
(86) PCT No.: PCT/EP2024/063298

§ 371 Date: Nov. 5, 2025

Related U.S. Application Data
(60) Provisional application No. 63/467,694, filed on May 19, 2023.

(30) Foreign Application Priority Data
Jul. 10, 2023 (EP) 23184355.8

(51) Int. Cl. A61N 1/36

(52) U.S. Cl. CPC A61N 1/36031

(57) ABSTRACT

A system of communicatively coupled nodes including at least two nodes. A first node of the at least two nodes includes a first sensor implantable and/or attachable to a patient for sensing the patient. A second node of the at least two nodes includes a second sensor for sensing the patient or a stimulator for stimulating the patient. The system is configured such that an event determined at the first node initiates an action at the second node.

### CROSS-REFERENCE TO RELATED APPLICATIONS

This application is the United States National Phase under 35 U.S.C. § 371 of PCT International Patent Application No. PCT/EP 2024/063298, filed on May 15, 2024, which claims the benefit of European Patent Application No. 23184355.8, filed on Jun. 10, 2023, and U.S. Provisional Patent Application No. 63/467,694, filed on May 19, 2023, the disclosures of which are hereby incorporated by reference herein in their entireties.

### TECHNICAL FIELD

The present invention relates to a system of communicatively coupled nodes and a respective method and computer program.

### BACKGROUND

A variety of medical devices have long been known that can be used for sensing and/or stimulating a patient. For example, a medical sensing device may be configured for sensing a particular physiological parameter of the patient. The characteristics of the sensed physiological parameter may then be used to assess the patient (e.g., for diagnostic purposes). In some cases, a sensing device may also be configured as a stimulation device (e.g., a cardiac stimulator) to stimulate the patient in view of the physiological parameter sensed by the device itself.

For medical or technical reasons, the sensing capabilities of a medical sensing device may be limited to monitor only one (or only few) physiological parameters of the patient. Also, for medical or technical reasons the stimulation capabilities of a medical stimulation device may be limited to a particular type of stimulation (e.g., a specific cardiac stimulation or a specific type of neurostimulation). Therefore, it is known to subject the patient to a medical setup of a variety of medical devices with each device being configured to perform a specific type of sensing and/or a specific type of stimulation. This approach may enable a broader range of sensing and/or stimulation capabilities for the patient. Furthermore, it may ensure a more detailed assessment of the patient in view of the broader range of available monitoring data.

However, the medical devices in known medical setups are typically configured to function as isolated devices, with little or no communication to other medical devices. In some examples, each medical device of the medical setup may even function as an autonomous entity. For example, each medical device may perform its own sensing and/or stimulating regardless of the sensing of other medical devices of the medical setup. However, such an approach may not always be optimal.

Hence, there is a need for improving medical device setups for patients.

The aspects described herein address the above need at least in part.

The present disclosure is directed toward overcoming one or more of the above-mentioned problems, though not necessarily limited to embodiments that do.

### SUMMARY

A first aspect relates to a system of communicatively coupled nodes comprising at least two nodes; wherein a first node of the at least two nodes comprises a first sensor implantable and/or attachable to a patient for sensing the patient; wherein a second node of the at least two nodes comprises a second sensor for sensing the patient or a stimulator for stimulating the patient; wherein the system is configured such that an event determined at the first node initiates an action at the second node.

Notably, the invention may comprise a system which may be provided as a platform. According to the invention, the system may comprise two or more communicatively coupled nodes. A node may comprise a sensor for sensing the patient (e.g., a sensor capable of sensing a physiological parameter of the patient). Such a node may also be referred to herein as a sensing node. A node may also be a node that comprises a stimulator for stimulating the patient. Such a node may also be referred to herein as a stimulation node. For example, the simulator may be configured to apply one or more electromagnetic pulses to the patient's body. A node may also be a node that comprises both, a sensor and a stimulator (which may be referred to herein as sensing and stimulation node). A node may for example comprise a device (e.g., a medical device) wherein the device may comprise the sensor and/or stimulator of the node. The nodes of the system may be communicatively coupled such that at least one node may be configured to receive (and process) a message that was sent from another node of the system. According to the invention, an event determined at one node of the general system may initiate an action at another node of the system.

For example, the first node of the system may comprise a monitoring and/or sensing device that comprises the first sensor of the first node. The first sensor may be a sensor for sensing one or more specific physiological parameters of the patient. Notably, the characteristics of the monitoring and/or sensing device comprising the first sensor may also be the characteristics described herein for the first sensor (and/or vice versa).

The first sensor may comprise an implantable sensor for implanting into the patient. The first implantable sensor may be positioned within the patient (e.g., as an implant). In this example, the first sensor may be configured to sense a physiological parameter from within the patient. The first sensor may, however, not necessarily be limited to an implant.

The first sensor may also comprise a sensor attachable to the patient. For example, the first attachable sensor may comprise a sensor that can be attached to the outside of the patient. The first sensor may thus, for example, be positioned on the patient's body and/or in a close vicinity to the patient's body. In this example, the first sensor may be configured to sense a physiological parameter from outside the patient. In an example, the first attachable sensor can be attached to the patient such that the patient may still move in an autonomous manner through an environment. For example, the first attachable sensor may be able to sense the patient without (necessarily) being connected via a wire to a stationary device.

For example, the second node of the first aspect may comprise a monitoring and/or sensing device that comprises the second sensor of the second node. The second sensor may be a sensor for sensing one or more specific physiological parameters of the patient. Notably, the characteristics of the monitoring and/or sensing device comprising the second sensor may also be the characteristics described herein for the second sensor (and/or vice versa).

For example, the second node may comprise a stimulation device that comprises the stimulator of the second node. The stimulator may be configured to apply one or more electromagnetic pulses to the patient. For example, the stimulator may comprise an electrode for applying the one or more electromagnetic pulse to the patient and a corresponding power circuit for generating the one or more electrical pulses. Notably, the characteristics of the stimulation device comprising the stimulator may also be the characteristics described herein for the stimulator (and/or vice versa).

In an example, the second node may comprise a sensing (and/or monitoring) and stimulation device that comprises the second sensor and the stimulator.

For example, the first node may be configured as a trigger node within the system. As described herein, an event determined at the first node may initiate an action at the second node. The first node may thus function within the system's architecture as a trigger node.

The second node may be configured as an action node within the system. As described herein, the event determined at the first node may trigger an action at the second node. The second node may thus function within the system's architecture as an action node.

Hence, according to the invention, a system of a medical setup can be provided wherein a dedicated node may be configured as a trigger node to allow for a responsive action of at least one other node of the system that is configured as an according action node. In this example the dedicated node comprises the first node which comprises a sensor implantable and/or attachable to a patient. The trigger, in this example, is caused by the event determined at the first node. This may be highly beneficial compared to common approaches that rely on various autonomous medical devices wherein each autonomous device performs an action based on a trigger event determined by the respective device itself (but not based on a separate implantable and/or attachable sensor configured as a trigger node).

For example, known medical setups may comprise stationary medical devices which, however, may not initiate an action based on an event determined by an implantable and/or attachable sensor of the patient.

Notably, the system of the first aspect may not be limited to two nodes. In an example, the system may comprise at least one additional node (e.g., a third node, a fourth node, etc.), wherein the at least one additional node comprises a further sensor for sensing the patient or a further stimulator for stimulating the patient; wherein the system is configured such that an event determined at the first node initiates an action at the at least one additional node.

The characteristics described herein for the second node, may also be accordingly applied to the at least one additional node. Notably, one or more additional nodes may be added to the system, wherein the characteristics described herein for the second node may be correspondingly applied to any additional nodes, as well. For example, if the system comprises a third node, the characteristics described herein for the second node may correspondingly apply to the characteristics of the third node. To illustrate another example, if the system comprises an additional third and fourth node, the characteristics described for the second node may be correspondingly applied to the characteristics of the third node and fourth node, respectively, as well.

In an example, the system may comprise at least three nodes, at least four nodes, at least five nodes, and/or at least ten nodes. Notably, not all nodes of the system have to be configured as trigger nodes or as action nodes. Some nodes of the system may also have a different functionality as described herein.

In an example, the second node may be implantable and/or attachable to the patient. For example, the second node may comprise the second sensor for sensing the patient. In such an example, the second sensor may comprise an implantable sensor for implanting into the patient. The second implantable sensor may be positioned within the patient (e.g., as an implant). In this example, the second sensor may be configured to sense a physiological parameter from within the patient. However, the second sensor may not necessarily be limited to an implant.

The second sensor of the second node may also comprise a sensor attachable to the patient. For example, the second attachable sensor may comprise a sensor that can be attached to the outside of the patient. The second sensor may thus, for example, be positioned on the patient's body and/or in a close vicinity to the patient's body. In this example, the second sensor may be configured to sense a physiological parameter from outside the patient. In an example, the second attachable sensor can be attached to the patient such that the patient may still move in an autonomous manner through an environment. For example, the second attachable sensor may be able to sense the patient without (necessarily) being connected via a wire to a stationary device.

For example, the second node may comprise the stimulator for stimulating the patient. In such an example, the stimulator may comprise an implantable stimulator for implanting into the patient. The implantable stimulator may be positioned within the patient (e.g., as an implant). In this example, the stimulator may be configured to stimulate the patient from within the patient's body. However, the stimulator may not necessarily be limited to an implant.

The stimulator of the second node may also comprise a stimulator attachable to the patient. For example, the stimulator may comprise a stimulator that can be attached to the outside of the patient. The stimulator may thus, for example, be positioned on the patient's body and/or in a close vicinity to the patient's body. In this example, the stimulator may be configured to stimulate the patient from outside the patient's body. In an example, the stimulator can be attached to the patient such that the patient may still move in an autonomous manner through an environment. For example, the stimulator itself may be able to stimulate the patient without (necessarily) being connected via a wire to a stationary device.

In an example, the event may be based at least in part on a physiological parameter of the patient sensed by the first node. For example, the event may comprise an event of the physiological parameter sensed at the first node. In an example, the event may correspond to a critical medical event of the physiological parameter (e.g., a sudden change of the physiological parameter that may be disadvantageous or unusual to the patient).

The first node may comprise processing capabilities for determining the event in view of the sensed physiological parameters. For example, the first node may comprise a processing module. The processing module may be configured to process the signals of the physiological parameter sensed by the first node. For example, the processing module may comprise a computing unit and/or one or more processors to perform various types of signal processing (e.g., performing a mathematical calculation, signal filtering, signal transformation, etc.). The processing module may also comprise an analog-to-digital converter to process an analog sense signal received from the first sensor.

In an example, the event may be determined based at least in part on the physiological parameter being below or above a predetermined threshold. For example, the first node may be configured to sense a physiological parameter A. An event may be associated with the physiological parameter A being below (or dropping below) a first predetermined threshold T1. Hence, the first node may be configured such that when A<T1 an event is determined by the first node. The according processing may be performed by the processing module of the first node.

In another example, the first node may be configured to sense a physiological parameter B. In this example, an event may be associated with the physiological parameter B being above (or shooting above) a second predetermined threshold T2. Hence, the first node may be configured such that when B>T2 an event is determined by the first node.

In another example, the first node may be configured to sense a physiological parameter C. In this example, an event may be associated with the physiological parameter C not residing in a certain range, for example, between a lower threshold T3 and an upper threshold T4. In this example, the first node may be configured such that when the condition T3<C<T4 is not met an event is determined by the first node.

In general, the first node may be configured to determine an event based at least in part on a defined change of a physiological parameters sensed at the first node.

In another example, the first node may be configured to determine an event based at least in part on a characteristic pattern of the physiological parameter. For example, the characteristic pattern may comprise a more complex signal pattern of the sensed signal of the physiological parameter (e.g., a shape of the signal, a frequency of the signal, a filter response, etc.). The characteristic pattern may also comprise that a physiological parameter is below or above a predetermined threshold for a specific time duration. For example, an event may only be determined if the predetermined threshold is crossed for the specific time duration. To that regard, the processing module of the first node may be configured to extract the characteristic pattern via an according signal processing.

In another example, the characteristic pattern, may comprise two or more subevents determined by the first node. In this example, an event that initiates an action at the second node would require that two or more subevents were determined at the first node. For example, a subevent may comprise a comparatively short event of the physiological parameter (e.g., a crossing of a predetermined threshold over a specific time duration, e.g., in the manner of a peak of the physiological parameter). To that regard, the subevent may not necessarily constitute a relevant (e.g., medically relevant) event. However, when multiple subevents may be determined over a predetermined time window at the first node, an event may be determined at the first node since a meaningful (e.g., medical) relevance may be given in this situation.

In another example, the first node may be configured to determine an event based at least in part on a defined change of two or more physiological parameters sensed at the first node. For example, this may be the case when the first sensor comprises a sensor for sensing two or more physiological parameters.

Subsequently, the action that can be initiated by the second node will be described. As described herein, the system may also comprise at least one additional node. Aspects, features, characteristics described with respect to the action of the second node may thus also correspondingly apply to the action of the at least one additional node.

In an example, the action may comprise storing, on the second node, data of a physiological parameter sensed by the second node. According to the invention, the event determined at the first node may cause in turn a storing of data on another node of the system. For example, the first node may determine an event based on the physiological parameter sensed by the first node. This event will cause the second node to store data of a physiological parameter sensed by the second node. Hence, the system of the first aspect may allow for a collective data storage of various physiological parameters of the patient throughout the system in view of a specific trigger event occurring at the first node.

In another example, the system may be configured such that an event determined at the first node may also initiate an action at the first node. Notably, the characteristics described herein for the initiated action at the second node, may also be accordingly applied to the initiated action at the first node.

For example, when an event is determined at the first node, the first node may store data of the physiological parameter sensed at the first node for a predetermined time duration, for example. When an event is determined at the first node, the first node may also store historical data of the physiological parameter sensed at the first node before the event was determined.

The second node (and/or the first node) may comprise a storage medium for storing the corresponding data (e.g., a memory, a memory storage unit). The second node may also comprise a processing module as described herein for the first node. The processing module of the second node may be configured to process the initiated action based on the determined event at the first node.

As described herein, the system may not necessarily be limited to two nodes. Hence, in another example, the action may comprise storing, on the additional node (e.g., on a third node, a fourth node, etc.), data of a physiological parameter sensed by the additional node (in the same manner as described herein for the second node).

In an example, the action may be initiated by a trigger sent from the first node to the second node. The trigger may comprise a trigger message sent from the first node to the second node. The trigger message may comprise a flag (e.g., a simple instruction to trigger an action, e.g., “TRIGGER” and/or a binary string). The trigger message may further comprise the node from which the trigger message was sent (e.g., to indicate that it was sent from the first node, e.g., the trigger message may comprise the information “TRIGGER from first node”). The trigger message may also comprise a more complex message and/or protocol. For example, the trigger message may comprise more complex trigger instruction. The trigger message may also comprise trigger information on the event at the first node that initiated the action. For example, the trigger information may comprise a value of the physiological parameter sensed by the first node and/or details on the condition that has caused the trigger.

In an example, the second node may store the trigger (e.g., the trigger message) at the second node. In an example, the trigger may not be (necessarily) sent directly from the first node to the second node. It may also be possible that the trigger is sent via a relay node of the system. For example, the trigger may be sent from the first node to the relay node, and then from the relay node to the second node. In other examples, there may be direct communication between the first and second nodes.

The communicative coupling of the nodes may be based on a wireless communication. For example, the first and second node of the system may be configured to communicate via radio frequency (RF). For example, the first node may comprise an RF sender, wherein the second node may comprise an RF receiver. Notably, the first node may also comprise an RF receiver, wherein the second node may also comprise an RF sender. In another example, the first and second node may both comprise RF transceivers.

In general, any node of the system may be configured for a wireless communication to another node of the system (e.g., via RF communication). In an example, the RF communication may, for example, comprise Bluetooth communication and/or Wi-Fi. However, it may also be conceivable that two or more nodes of the system communicate via a wire.

In another example, two or more nodes of the system (e.g., the first and second node and/or the additional node) may be configured for a communication via conducted (electrical) pulses within and through the patient's body. In another example, two or more nodes of the system may be configured for ultrasound communication, acoustic communication and/or telemetric communication.

In an example, the action may further comprise: sensing, by the second node, the data of the physiological parameter after the trigger was received for a predetermined time period; and/or storing, by the second node, historical data of the physiological parameter sensed by the second node before the trigger was received.

In this example, upon receiving of the trigger the second node may initiate a sensing of the data of the physiological parameter that can be sensed at the second node. The sensing may take place over a predetermined time period. The sensed data may then be stored on the second node (e.g., as described herein). Hence, the trigger may initiate a recording, by the second node, of the data of the physiological parameter sensed at the second node. The recorded data may be stored with the information that the trigger of the first node has caused the recording. The predetermined time period for sensing may be a fixed time period chosen for the second node, for example. The predetermined time period may also depend on the trigger and/or on the first node that has send the trigger (e.g., a device type of the first node, a type of physiological parameter of the first node).

The recording of the data at the second node based on the trigger of the first node may be highly beneficial. For example, the first node may sense a first physiological parameter, wherein the second node may sense a second physiological parameter (different from the first physiological parameter). The first node may determine an event for the first physiological parameter such that the second node starts recording the second physiological parameter. Hence, the event may be analyzed in view of two different physiological parameters of the patient. This may enable a more holistic analysis of the patient's condition associated with the event. Moreover, the effects the event may induce on the patient's body may be better assessed based on the data of the two different physiological parameters.

This concept of triggered data storage may also enable saving of data storage. According to the invention, relevant data of the second node may only be stored when a trigger from the first node is received. A continuous recording of the physiological parameter at the second node may thus not be necessary. Unnecessary data thus does not need to be stored by the second node.

In another example, the first node and the second node may sense the same physiological parameter. In this case, the recorded data may be used to cross-check or verify the sensed physiological parameter of the first and second node.

Furthermore, the example may also comprise a storing of historical data on the second node upon receiving the trigger from the first node. For example, the second node may be configured to record data over a certain recording window (e.g., for the last minute, the last hour, the last day, etc.). However, the second node may thus also be configured to delete data outside of the recording window if no special indication is met of the physiological parameter sensed by the second node. This may be done to save storage space on the second node.

The invention may enable that the trigger of the first node may also cause storing historical data of the second node of particular relevance. This may be highly beneficial as this enables a data collection of physiological parameters leading up to the point when the event at the first node occurred. To illustrate an example, the first node may sense a first physiological parameter, wherein the second node may sense a second physiological parameter (different from the first physiological parameter). By storing historical data on the second node based on the trigger the event of the first physiological parameter may also be analyzed in view of historical characteristics of the second physiological parameter at the second node. This may enable a more holistic analysis of the patient's condition. Moreover, the causes of the event may be better assessed based on the data of the two different physiological parameters.

Namely, when the first and second node may sense the same physiological parameter, the historical data may also be used to cross-check or verify the sensed physiological parameter at the first and second node.

In an example, the action may further comprise analyzing, by the second node, the stored data for determining a status of the patient. For example, the second node may comprise a processing module for analyzing the stored data. The processing module may perform various types of signal processing and/or statistics to analyze the stored data for determining the status of the patient.

In an example, the second node may be configured to retrieve data of the sensed physiological parameter at the first node which is associated with the determined event (as described herein). As exemplarily illustrated herein, the first node may sense a first physiological parameter, wherein the second node may sense a second physiological parameter (different from the first physiological parameter). Hence, the second node may be configured to analyze the patient based on two different physiological parameters (e.g., the first and second physiological parameter) wherein an event has occurred for the first physiological parameter.

In an example, at least one node of the system may be configured as an analysis node for analyzing the data stored on the second node (and/or the first node) for determining a status of the patient. Hence, the analysis may also be performed by a node other than the second node. For example, an additional node of the system and/or the first node may be configured as an analysis node which may perform the analysis of the stored data. The analysis node may be configured to retrieve the stored data from the second and/or first node (or an additional node) of the system (e.g., via a communication to the respective node).

In an example, the analysis node may comprise an external computer which is integrated to the system as a communicatively coupled node.

In an example, the action may comprise stimulating the patient and/or adapting at least one stimulation parameter of the stimulator. For example, this action may be performed when the second node comprises a stimulator for stimulating the patient. As described herein, the stimulator may comprise a stimulator for applying one or more electromagnetic pulses to the patient. For example, the stimulator may comprise a cardiac stimulator (e.g., an implantable cardiac stimulator). The determined event at the first node may initiate that the cardiac stimulator starts to perform a specific stimulation sequence. The determined event at the first node may also initiate that a stimulation parameter of the cardiac stimulator is adapted or changed (e.g., a stimulation rate, a stimulation delay between two simulation pulses, etc.).

In an example, the first node may be further configured such that it may initiate an action based at least in part on an event determined at another node of the system for sensing and/or stimulating the patient. In this example, the first node may thus be configured as an action node of the system, as well. The first node may thus not be limited to function as a trigger node.

In an example, the second node may further be configured as a trigger node. In this example, the system may be configured such that an event determined at the second node initiates an action at the first node. The actions described herein for the second node may thus also be accordingly applied to the first node, as well.

In summary, any node of the system may be configured as a trigger node and/or as an action node. Nodes may be added to the system and/or removed from the system. However, not all nodes of the system must be configured as trigger and/or action nodes. Namely, the system may also comprise one or more nodes that are configured as analysis nodes (or relay nodes). In an example, an analysis node may also be configured as a trigger node and/or as an action node.

In an example, the first sensor attachable to the patient may comprise a wearable device that can be worn by the patient, preferably around a limb and/or a body part. For example, the wearable device may comprise any wearable technology device which may comprise a sensor for sensing the patient. The wearable device may, for example, comprise a patch that can be worn on the patient's body. The wearable device may, for example, comprise a device that can be worn around a wrist of the patient. For example, the wearable device may comprise a wearable watch (e.g., a smart watch) and/or a wrist band (e.g., a smart fitness bracelet).

The wearable device may also comprise an eye-worn device, a head-worn device, a foot-worn device and/or a device worn around finger of the patient (e.g., a ring, a glove, etc.). The wearable device may also comprise a device for attaching to an ear of the patient and/or a belt.

In another example, the wearable device may comprise any type of clothing that can be worn by a patient, wherein the clothing may comprise a sensor for sensing the patient.

In an example, the first and/or second sensor may comprise a sensor for sensing at least one of the following physiological parameters of the patient: an oxygen saturation, a blood oxygen saturation, a blood pressure, an electrocardiogram, a motion and/or movement, a posture, a respiration. For example, the first and/or second sensor may comprise a sensor for sensing a peripheral oxygen saturation (SPO₂) of the patient.

In an example, the first sensor may comprise an oxygen saturation sensor, wherein the second sensor may comprise a blood pressure sensor and/or a sensor for sensing an electrocardiogram of the patient.

In an example, the first sensor may comprise a blood pressure sensor and/or a sensor for sensing an electrocardiogram of the patient, wherein the second sensor may comprise an oxygen saturation sensor.

In an example, the first sensor may comprise a motion sensor, wherein the second sensor may comprise a blood pressure sensor and/or an oxygen saturation sensor.

In example, the system may comprise a system of at least three nodes with a first additional node being comprised by the system besides the first and second node. In this example, the first sensor of the first node may comprise an oxygen saturation sensor. The second sensor of the first node may comprise a blood pressure sensor. At least one of the first and second node may be configured as a trigger node. At least one of the first and second node may be configured as an action node. The first additional node may comprise a sensor for sensing an electrocardiogram of the patient (e.g., an implantable and/or an attachable electrocardiogram sensor) and/or a stimulator for stimulating the patient (e.g., an implantable and/or attachable stimulator). The first additional node may be configured as an action node and/or as a trigger node within the system. In another example, the system may further comprise a second additional node. The second additional node may comprise a sensor for sensing a respiration and/or motion of the patient (e.g., an implantable and/or attachable respiration sensor, e.g., an implantable and/or attachable motion sensor). The second additional node may be configured as an action node and/or as a trigger node within the system.

To illustrate an example, in the described exemplary systems with at least three nodes every node may function as a trigger node and as an action node. For example, a critical drop in the oxygen saturation determined as a (trigger) event at the first node may initiate an action in the other system nodes. This may, for example, comprise recording of the blood pressure, recording of an electrocardiogram, recording of respiratory data and/or recording of a motion of the patient by the respective nodes. Furthermore, this may also comprise storing historical data of the blood pressure, the electrocardiogram, the respiratory data and/or the motion of the patient by the respective nodes. Also, the (trigger) event may cause a specific stimulation by the respective (first additional) node. Notably, any other event determined by another node of the system may function as a (trigger) event for the system.

The system architecture of the invention may thus enable a high degree of freedom for analyzing and/or treating a patient. The stored data may be retrieved by a node for assessing a status of the patient, for example, for diagnostic purposes.

In an example, the status of the patient that may be determined by the system comprises at least one of the following: a cardiac arrythmia, a sleep apnea, a syncope. However, determining the status may not be (necessarily) limited to determining a health condition, as such. For example, determining the status may also comprise determining a specific type of health condition of the patient (e.g., a specific type of cardiac arrythmia, a specific type of sleep apnea, a specific type of syncope).

A second aspect relates to a method for performing in a system of communicatively coupled nodes, the system comprising at least two nodes; wherein a first node of the at least two nodes comprises a first sensor implantable and/or attachable to a patient for sensing the patient; wherein a second node of the at least two nodes comprises a second sensor for sensing the patient or a stimulator for stimulating the patient; the method comprising: initiating an action at the second node based at least in part on an event determined at the first node.

In an example, the method may comprise configuring the first node as a trigger node within the system (as described herein). In another example, the method may comprise configuring the second node as an action node within the system (as described herein).

A third aspect relates to a computer program comprising instructions that when executed by a computer cause the computer to perform a method according to the second aspect. In an example, the third aspect may relate to a (non-transitory machine readable) storage medium comprising the instructions of the computer program.

A fourth aspect relates to the first node as described herein that is configured for a system according to the first aspect.

A fifth aspect relates to the second node as described herein that is configured for a system according to the first aspect.

It is noted that the method steps (or computer program steps) as described herein may include all aspects described herein, even if not expressly described as method steps but rather with reference to a system (or device or apparatus). Moreover, the devices (or systems) as outlined herein may include means for implementing all aspects as outlined herein, even if these may rather be described in the context of method steps.

Whether described as method steps, computer program and/or means, the functions described herein may be implemented in hardware, software, firmware, and/or combinations thereof. If implemented in software/firmware, the functions may be stored on or transmitted as one or more instructions or code on a computer-readable medium. Computer-readable media include both computer storage media and communication media including any medium that facilitates transfer of a computer program from one place to another. A storage medium may be any available medium that can be accessed by a general purpose or special purpose computer. By way of example, and not limitation, such computer-readable storage media can comprise RAM, ROM, EEPROM, FPGA, CD/DVD or other optical disk storage, magnetic disk storage or other magnetic storage devices, or any other medium that can be used to carry or store desired program code means in the form of instructions or data structures and that can be accessed by a general-purpose or special-purpose computer, or a general-purpose or special-purpose processor.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

FIG. 1 shows a first exemplary system according to the invention.

FIG. 2 shows a second exemplary system according to the invention.

### DETAILED DESCRIPTION

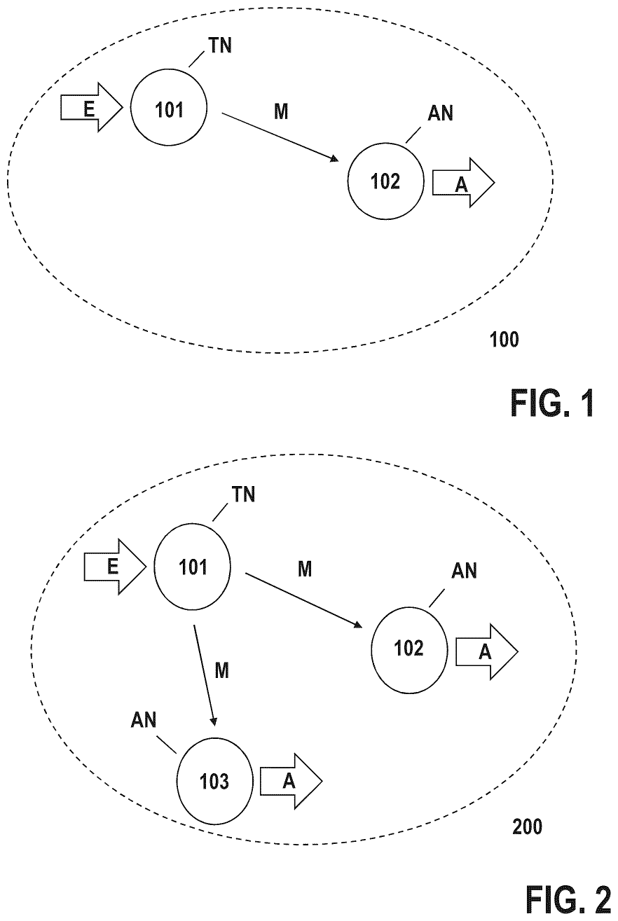

The first exemplary system 100 according to the invention shown in FIG. 1 comprises two nodes. Namely, the system 100 comprises a first node 101 and a second node 102. The first node 101 may correspond to the first node of the invention as described herein. The second node 102 may correspond to the second node of the invention as described herein.

The first node 101 of the system 100 may comprise a first sensor for sensing a patient. The first node 101 may thus function as a sensing node of the system. The first sensor of the first node 101 may comprise a sensor implantable within a patient. In another example, the first sensor of the first node 101 may comprise a sensor attachable to a patient. The first sensor of the first node 101 may be configured to sense a first physiological parameter of the patient. The first node 101 may comprise a first processing module (e.g., as described herein). The first node 101 may further comprise a first storage medium (e.g., as described herein).

In the example of FIG. 1, the first node 101 may be configured as a trigger node TN within the system 100. Hence, the first node 101 may be configured to determine an event E of the sensed first physiological parameter (e.g., via the first processing module). For example, the event E may comprise that the first physiological parameter crosses a predetermined threshold.

The second node 102 of the system 100 may comprise a second sensor for sensing a patient. In such an example, the second node 102 may thus function as a sensing node of the system 100. The second sensor of the second node 102 may comprise a sensor implantable within a patient. In another example, the second sensor of the second node 102 may comprise a (wearable) sensor attachable to a patient. The second sensor of the second node 102 may be configured to sense a second physiological parameter of the patient (different from the first physiological parameter). The second node 102 may comprise a second processing module (e.g., as described herein). The second node 101 may further comprise a second storage medium (e.g., as described herein).

The second node 102 of the system 102 may also comprise a stimulator for stimulating a patient. In such an example, the second node 102 may thus function as a stimulation node of the system 100. The stimulator of the second node 102 may comprise a stimulator implantable within a patient. In another example, the stimulator of the second node 102 may comprise a (wearable) stimulator attachable to a patient.

In the example of FIG. 1, the second node 101 may be configured as an action node TN within the system 100. Hence, the second node 101 may be configured to initiate an action A based at least in part on an event E determined by another node of the system.

Subsequently, an exemplary mode of operation of the first system 100 is explained.

The first node 101 may determine that an event E has occurred for the first physiological parameter (e.g., via the first processing module).

Upon determining the event E, the first node 101 may start recording the first physiological parameter. The recording of the first physiological parameter may comprise a recording of the event E and/or a subsequent course of the first physiological parameter for a predetermined time window (e.g., for 10 s for 1 min, for 30 min, for 1 hour etc.). Upon determining the event E, the first node 101 may also start storing historical data of the first physiological parameter on the storage medium of the first node 101 preceding the event E. For example, the historical data may comprise a course of the first physiological parameter of the last 10 s, the last 1 min, the last 2 min, the last 10 min, the last 30 min, the last 1 hour, etc. preceding the even E.

Notably, the first node 101 may be configured as a trigger node TN within the system 100. Hence, upon determining the event E, the first node 101 may send a trigger message M throughout the first system 100. The trigger message M may comprise an instruction that will cause another node of the system configured as an action node AN to initiate a corresponding action A.

In the example of FIG. 1, the trigger message M may be received by the second node 102. Notably, the second node 102 may be configured as an action node AN within the system 100. Hence, upon receiving the trigger message M the second node 102 may initiate a corresponding action A.

The action A at the second node 102 may comprise starting a recording of the second physiological parameter for a predetermined time period (e.g., for 10 s, for 1 min, for 30 min, for 1 hour, etc.). The action A at the second node 102 may also comprise storing historical data of the second physiological parameter on the storage medium of the second node 102 (e.g., the historical data of the last 10 s, the last 1 min, the last 2 min, the last 10 min, the last 30 min, the last 1 hour, etc.).

The event E at the first node 101 may thus cause the system 100 to record/store data of two physiological parameters which are sensed by two different sensors (of two different devices).

The system 100 may thus enable a defined integration of multimodal physiological data of various sensors. For example, the event E may thus be assessed in view of the first and second physiological parameter stored on the system. The inventive system 100 may thus be used to combine vital records and diagnostic data among a multitude of devices. This may ensure an improved diagnosis of the patient and/or an improved screening for high-risk scenarios of the patient. The analysis of the recorded/stored data may be performed by the first node 101 and/or the second node 102. In an example, the analysis may be performed by an analysis node of the system 100 (for example an external computer).

Furthermore, the system 100 may enable the ability to trigger one or more types of data storage in view of a determined event E such that data storage is focused on the time periods and/or events of importance. Moreover, the battery life and/or longevity of the system nodes may thus be improved, as an action A may only be required during periods and/or events of importance.

In addition, the system 100 may enable an ability to reduce excessive data volume within the system's nodes. Therefore, efficiency and workflow for clinical personnel may be improved.

As described herein. the second node 200 may comprise a stimulator. The action A initiated at the second node 200 may thus also comprise starting a stimulation via the stimulator. Additionally and/or alternatively the action A may thus also comprise adapting at least one stimulation parameter of the stimulation via the stimulator.

It should be noted that the first node 101 may also be configured as an action node AN and the second node 102 may also be configured as a trigger node TN. Hence, an event E determined at the second node 102 may cause the second node 102 to send a trigger message M throughout the system 100 to the first node 101. Subsequently, the first node may initiate an action A upon receiving the trigger message M.

As described herein, one or more additional nodes may be added to the system 100 that comprises two nodes. The herein described system architecture of configuring nodes as trigger and/or action nodes may allow for an easy and defined integration of additional nodes (e.g., devices, sensors, stimulators).

In FIG. 2 a second exemplary system 200 according to the invention is shown which, for example, comprises three nodes. The system 200 may comprise the first node 101 and second node 102 of the first exemplary system 100. However, the system 200 has been extended by a third node 103 (an additional system node). All characteristics described herein for the second node may be accordingly applied to the third node 103.

For example, the third node may comprise a third sensor for sensing a third physiological parameter. Notably, the third node 103 may be configured as an action node AN just as the second node 102. Hence, when the event E is determined at the first node 101 the trigger message M sent from the first node 101 may be received by the third node 103. Upon receiving the trigger message M, the third node 103 may initiate an action A (in a similar manner as described for the second node). For example, the third node 103 may start recording the third physiological parameter and/or storing historical data of the third physiological parameter.

The third node 103 may also comprise a stimulator, wherein the trigger message M may initiate a stimulation via the third node 103 and/or adapting a stimulation parameter of the third node 103.

Moreover, the third node 103 may be configured as a trigger node TN of the system 200. Hence, an event E determined at the third node 103 may cause the third node 103 to send a trigger message throughout the system 200. Subsequently, the action nodes AN of the system 200 receiving said trigger message M may start initiating their corresponding actions (as described herein).

Notably, various additional nodes may be added to the system 200. For example, the system 200 may also comprise a fourth node, a fifth node and/or a sixth node (as described herein).

Subsequently, various system application examples according to the invention will be explained in more detail. The examples may comprise examples of the first system 100 (if the example only requires two nodes). The examples may also comprise examples of the second system 200 (if the example comprises three or more nodes). Subsequently, the nodes will be described within their functionality of trigger nodes and/or action nodes to account for the multimodal character of the invention.

A first system application example may comprise sensing an oxygen saturation (e.g., SpO₂) via a trigger node. Such a trigger node may be referred to herein as a SpO₂ trigger node. The SpO₂ trigger node may, in this example, comprise a device that can be worn on the wrist, in a band around a limb and/or body part. The SpO₂ trigger node may also comprise a device that can be worn as a patch somewhere on the body.

The SpO₂ trigger node may be configured to determine as an event E whether a drop in the oxygen saturation (e.g., in a SpO2 value) occurs. The condition for detecting the event may be chosen such that the event E may indicate a pathological condition or a condition of high risk. According to the invention other sensors of the system may then be activated as action nodes A. For example, when the SpO₂ value drops below 90% a trigger message M may be sent to one or more action nodes of the system. In this example, an action node may comprise an implantable device which comprises a sensor for sensing an electrocardiogram of the patient. Such an action node may be referred to herein as an EKG action node. Upon receiving the trigger message M, the EKG action node may begin recording an electrocardiogram (ECG or EKG) of the patient from that point forward. Upon receiving the trigger message M, the EKG action node may also store historical data of the electrocardiogram up until the trigger message was received.

In the first application example, the SpO₂ drop may thus trigger data storage of an electrocardiogram of the patient within the system. This information may be used to analyze what caused the drop in SpO₂ and/or to monitor the consequences of that drop (e.g., the consequences in the electrocardiogram signal).

In the first application example, the trigger message M associated with SpO₂ drop may also trigger other sensors of the system that are configured as action nodes. For diagnostic purposes it may be of high interest to record other physiological parameters preceding and following the drop in SpO_{2.}

For example, another action node may comprise a sensor for sensing respiration activity. Such an action node may be referred to herein as a respiration action node. The SpO₂ drop may thus trigger recording/storing of the respiration activity at the respiration action node before and/or after the change in SpO₂ has occurred.

For example, another action node may comprise a sensor for sensing a blood pressure of the patient. Such an action node may be referred to herein as blood pressure action node. The SpO₂ drop may thus trigger recording/storing of the blood pressure at the blood pressure action node before and/or after the change in SpO₂ has occurred. Notably, blood pressure may be highly related to an SpO₂ characteristic.

In general, any data could be stored as a result of the SpO₂ trigger within an action node of the system.

A second system application example may comprise sensing a blood pressure via a trigger node. Such a node may also be referred to herein as a blood pressure trigger node of a system according to the invention. The blood pressure trigger node may comprise an implantable device for implanting in a pulmonary artery. The blood pressure trigger node may also comprise a wearable device worn on the body (and/or a sensor located in another location). The blood pressure trigger node may determine an event E based on a critical change in blood pressure and send out an according trigger message M. This event E may thus trigger the recording/storing of data at action nodes of the system before and/or after the change in blood pressure has occurred (as described herein).

For example, the blood pressure change may trigger recording/storing of sensed SpO₂ values at an SpO₂ action node of the system before and/or after the change in blood pressure has occurred. Notably, the herein described SpO₂ trigger node may also be configured as an SpO₂ action node.

For example, the blood pressure change may trigger recording/storing of an electrocardiogram at an EKG action node of the system before and/or after the change in blood pressure has occurred.

For example, the blood pressure change may trigger recording/storing of an respirational activity at an respiration action node of the system before and/or after the change in blood pressure has occurred.

For example, the blood pressure change may trigger recording/storing of sensed motional activity at a motion action node of the system before and/or after the change in blood pressure has occurred. The motion action node may comprise a motion sensor for sensing a motion of the patient.

The data from the action nodes (and/or the recorded/stored data of the blood pressure trigger node) may be used alone or in combination to determine the cause. The data may also be used to diagnose the pathology associated with the change in blood pressure.

A third system application may comprise using a trigger node that comprises a motion sensor (i.e., a motion trigger node). The motion trigger node may comprise an implant and/or a wearable device. The event E determined by the motion trigger node may be a fall of the patient (or a sudden critical change in posture of the patient). The motion trigger node may thus be used to assess a syncope of the patient (associated with a fall of the patient). In such an example, the fall of the patient may (in particular) b related to a change in blood pressure.

A change in motion (e.g., a fall) determined by the motion trigger node may thus, for example, trigger recording/storing of the sensed blood pressure at a blood pressure action node.

However, a change in motion may also trigger recording/storing in other action nodes (e.g., in an SpO₂ action node, an EKG action node, a respiration action node, a motion action node etc.). Hence, the change in motion may trigger a recording/storing of SpO₂ values, of an electrocardiogram, respirational and/or motional activity. For instance if motion trigger node detects a fall of the patient, the data before and after that event would be recorded to determine if there was a precipitating event in the EKG, in the SpO₂ or in the blood pressure, for example.

A fourth system application may comprise triggering an action at an action node of the system that comprises a stimulator. For example, such an action node may be referred to herein as a stimulation action node. In such an example, a trigger node may thus trigger a specific sequence at the stimulation action node (e.g., starting a stimulation and/or adapting at least one stimulation parameter of the stimulator). For example, a blood pressure drop or SpO₂ drop (determined by according trigger nodes) may trigger an action at the stimulation action node (as this may be of medical significance for the patient). However, in general, also other trigger nodes may trigger an action at the stimulation action node.

In an example, the stimulator of the stimulator action node may comprise a cardiac stimulator (e.g., an implantable cardiac stimulator, e.g., an implantable pacemaker).

For example, an event determined at a trigger node may cause adapting a stimulation rate of the cardiac stimulator (e.g., a pacing rate). To illustrate another example, an event determined at a trigger node may cause adapting a pacing and/or sensing delay of the cardiac stimulator (e.g., a delay between an atrial sense and a ventricular pace, a delay between an atrial pace and a ventricular pace, a delay between an atrial sense and a ventricular sense). For example, the adapted pacing and/or sensing delay of the cardiac stimulator may comprise an AV delay. It may also be conceivable, that an event determined at a trigger node may cause adapting parameters of the cardiac stimulator that relate to a (stimulation/pacing) rate response, a (stimulation/pacing) rate fading, or a (stimulation/pacing) rate increase. In summary, various pacing/stimulation parameters of the cardiac stimulator having medical relevance may be adapted based on a trigger send from a trigger node of the system. Notably, it may be of medical relevance that in particular a drop in blood pressure (determined at a blood pressure trigger node) may trigger an according sequence at the cardiac stimulator.

In another example, the stimulator may comprise at least one of the following: an implantable pulse generator (IPG), an implantable cardiac defibrillator (ICD).

In an example, a sensor (e.g., the first and/or second sensor) may comprise an implantable cardiac monitor (ICM) which may be used to sense an electrocardiogram of the patient.

A fifth system application may be tailored to assess sleep apnea. For example, oxygen saturation, respiration data, and an electrocardiogram of the patient can be used together in order to assess sleep apnea. For instance, in obstructive sleep apnea there may be chest wall movement related to respiratory effort but an obstruction prevents airflow. Thus, the oxygen saturation will fall while chest wall motion remains intact. In contrast central sleep apnea may be characterized by the cessation of all respiratory effort and therefore no chest wall motion or minimal chest wall motion with a simultaneous drop in oxygen saturation.

These relationships can be used to differentiate the types and severity of sleep apnea. The herein described system may thus serve as a suitable platform for triggering the suitable recoding/storing of data of the according sensors (configured as the systems action and/or trigger nodes).

For example, a suitable trigger for apnea events may comprise an event determined in at least one of the following physiological parameters: respiration activity, motion, posture, oxygen saturation (e.g., SpO₂). However, also other physiological parameters may be used as triggers for a sleep apnea event. Moreover, such a trigger may be used for any action node (as described herein). For example, a sleep apnea trigger may cause a recording/storing of data of an electrocardiogram, a motion, a posture, a blood pressure, an oxygen saturation (as described herein).

For example, an electrocardiogram may be used to determine causes of an apnea. For example, the apnea may be related to a cardiac arrythmia of a patient wherein the causing arrythmia may be analyzed via stored historical data of an electrocardiogram preceding the apnea trigger. Moreover, the triggered electrocardiogram recording may also be used to assess the impact of an apnea such as an increased arrhythmias following apnea events.

The assessment of sleep apnea may be even further improved when the system is adapted such that a sleep apnea trigger may initiate a recording/storing of motion and/or posture of the patient. This data may, for example, be analyzed in combination with the electrocardiogram data (and/or blood pressure data and/or oxygen saturation data).

For example, the combination of recorded/stored motion sensor data comprising a posture information would confirm sleeping position, restlessness, quality of sleep which may be relevant to the diagnosis and/or tracking of sleep apnea.

The invention may thus be highly advantageous for diagnostic purposes as it provides the possibility to interlink various sensing devices via a defined system protocol. This may allow to bring multiple physiologic data together synchronously in order to enable an improved diagnosis of the condition of the patient. As described herein, the system may allow a holistic analysis of hemodynamic data from blood pressure, oxygenation data from SpO₂ data, and cardiac rhythm data from an electrocardiogram.

In addition, the multimodal data collection is streamlined as it its only triggered based on sensed events. This may avoid the problem of too much data within the system.

Furthermore, the system architecture is designed towards patient comfort as the nodes of the system may rather comprise implants and/or wearable devices. The patient may thus be monitored (and/or stimulated) via the system independent of a stationary clinical setting.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. System of communicatively coupled nodes comprising at least two nodes;
wherein a first node of the at least two nodes comprises a first sensor implantable and/or attachable to a patient for sensing the patient;
wherein a second node of the at least two nodes comprises a second sensor for sensing the patient or a stimulator for stimulating the patient;
wherein the system is configured such that an event determined at the first node initiates an action at the second node.

2. System according to claim 1, wherein the second node implantable and/or attachable to the patient.

3. System according to claim 1, wherein the event is based at least in part on a physiological parameter of the patient sensed by the first node.

4. System according to claim 1, wherein the at least two nodes are communicatively coupled via wireless communication.

5. System according to claim 1, wherein the action comprises storing, on the second node, data of a physiological parameter sensed by the second node.

6. System according to claim 1, wherein the action is initiated by a trigger sent from the first node to the second node.

7. System according to claim 6, wherein the action further comprises at least one of the following:
sensing, by the second node, the data of the physiological parameter after the trigger was received for a predetermined time period; and/or
storing, by the second node, historical data of the physiological parameter sensed by the second node before the trigger was received;

8. System according to claim 5, the action further comprising analyzing, by the second, the stored data for determining a status of the patient.

9. System according to claim 5, wherein at least one node of the system is configured as an analysis node for analyzing the data stored on the second node for determining a status of the patient.

10. System according to claim 1, wherein the action comprises stimulating the patient and/or adapting at least one stimulation parameter of the stimulator.

11. System according to claim 1, wherein the first node is further configured such that it may initiate an action based at least in part on an event determined at another node of the system for sensing and/or stimulating the patient.

12. System according to claim 1, wherein the first sensor attachable to the patient comprises a wearable device that can be worn by the patient, preferably around a limb and/or a body part.

13. System according to claim 1, wherein the first and/or second sensor comprises a sensor for sensing at least one of the following physiological parameters of the patient: an oxygen saturation, a blood oxygen saturation, a blood pressure, an electrocardiogram, a motion and/or movement, a posture, a respiration.

14. Method for performing in a system of communicatively coupled nodes, the system comprising at least two nodes;
wherein a first node of the at least two nodes comprises a first sensor implantable and/or attachable to a patient for sensing the patient;
wherein a second node of the at least two nodes comprises a second sensor for sensing the patient or a stimulator for stimulating the patient;
the method comprising:
initiating an action at the second node based at least in part on an event determined at the first node.

15. Computer program comprising instructions that when executed by a computer cause the computer to perform a method according to claim 14.
